(19) United States
(12) Patent Application Publication
(10) Pub. No.: US 2010/0249617 A1
(43) Pub. Date: Sep. 30, 2010

(54) Apparatus for determining blood pressure

(75) Inventors: Lap Wai Lydia Leung, Hong Kong (CN); Kwan Wai Raymond To, Tuen Mun (HK); Chi-Tin Luk, Tuen Mun (HK); Chang Hwa Tom Chung, Shatin (HK)

Correspondence Address: LEYDIG VOIT & MAYER, LTD, 700 THIRTEENTH ST. NW, SUITE 300, WASHINGTON, DC, 20005-3960, US

(73) Assignee: Hong Kong Applied Science and Technology Research Institute Company Limited, Shatin (HK)

(21) Appl. No.: 12/415,280

(22) Filed: Mar. 31, 2009

(51) Int. Cl. A61B 5/0225

(52) U.S. Cl. 600/495

(57) ABSTRACT

An apparatus for determining the blood pressure of a patient which allows the patient to first measure his measured blood pressure using a blood pressure measuring device and then, at a later time, estimate his current blood pressure using a blood pressure calculating device that includes a mathematical model for estimating blood pressure based on a current change in blood pressure induced by a heart beat and the previously measured blood pressure. The apparatus includes a transmitter device that detects the presence of a blood pressure calculating device connected to or in proximity of the blood pressure measuring device and transmits the previously measured blood pressure value to the blood pressure calculating device.

### FIELD OF THE INVENTION

The invention generally relates to an apparatus for determining the arterial blood pressure of human, and in particular to an apparatus that automatically calibrates a blood pressure calculating device.

### BACKGROUND TO THE INVENTION

Blood pressure is a fundamental indicator of a person's health. High blood pressure (hypertension) can be caused by such factors as genetics, obesity, lack of exercise, overuse of salt, and aging. Prolonged hypertension increases the risk of developing heart and kidney disease, hardening of the arteries, eye damage and stroke. Both high and low blood pressure can also be indicators of heart disease. It is therefore advantageous for people to be able to simply and accurately measure their own blood pressure at home, work or on holiday, for example.

As a person's heart beats their blood pressure rises and falls between a static or diastolic pressure and a maximum or systolic pressure within the arteries. Blood pressure is expressed as systolic pressure over their diastolic pressure, for example 140/90. The most common way of measuring blood pressure is using a sphygmomanometer. In a manual sphygmomanometer an inflatable cuff is wrapped around the upper arm of a person and inflated until the level of mercury in a connected barometer reaches a predetermined threshold at which blood flow in the brachial artery stops. A stethoscope is need for the detection of the Korotkoff sounds. The head of the stethoscope is placed over the brachial artery and air is slowly released from the cuff. The Korotkoff sounds are detected while the cuff is being slowly deflated. The five Korotkoff sounds are a tapping sound, a soft swishing sound, a crisp sound a blowing sound and silence. The cuff pressures at which a tapping sound and silence are detected represent the systolic and diastolic blood pressures of the subject respectively.

A major problem with a cuff based blood pressure measuring device is that it is bulky and it takes some skill to use. Although automated cuff blood pressure measuring devices are available they remain bulky and noisy due to the pump for inflating the cuff. They are therefore not suitable for discrete, frequent or continuous measurement of blood pressure.

Blood pressure can also be estimated from pulse transit time. Arterial blood pressure is found to be inversely proportional to the velocity of a pulse wave in blood caused by a heart beat. This theory has been disclosed in some papers: Messrs J. C. Bramwell and A. V. Hill, “The Velocity of the Pulse Wave in Man”, Proceedings of the Royal Society, London, pp. 298-306, 1922; and B. Gribbin, A. Steptoe, and P. Sleight, “Pulse Wave Velocity as a Measure of Blood Pressure Change”, Psychophysiology, Vol. 13, No. 1, pp. 86-90, 1976, which are incorporated herein as reference. Pulse transit time can be calculated by measuring heart beat and blood flow perimeters using discrete electronic sensors and so is much more suitable for discrete, frequent or continuous determination of blood pressure.

However, a problem with the estimation of blood pressure via pulse transit time is that the relationship between pulse transit time and systolic and diastolic blood pressure changes with the physiological state of the person and so pulse transit time blood pressure estimating devices require frequent calibration in which blood pressure is simultaneously measured via a conventional cuff based blood pressure measuring device and by a pulse transit time method to establish an up-to-date relationship between pulse transit time and systolic and diastolic pressure.

### SUMMARY OF THE INVENTION

It is an object of the present invention to provide an apparatus for determining the arterial blood pressure of human, and in particular an apparatus that automatically calibrates a blood pressure calculating device, which ameliorates the above mentioned disadvantages, or which simply provides the public with a useful alternative.

There is disclosed herein an apparatus for determining the blood pressure of a patient which allows the patient to first measure his blood pressure using a blood pressure measuring device and then at a later time estimate his current blood pressure using a blood pressure calculating device that includes a mathematical model for estimating blood pressure based on a current change in blood volume induced by a heart beat and the previously measured blood pressure. The apparatus includes a transmitter device that detects the presence of the blood pressure calculating device connected to or in proximity of the blood pressure measuring device and transmits the previously measured blood pressure value to the blood pressure calculating device.

The blood pressure measuring device includes a conventional inflatable cuff that restricts blood flow for the measurement of systolic and/or diastolic blood pressure. It does use a mathematical model or calculation that includes a change in arterial blood induced by heart beat and the measured blood pressure value. Only the measured blood pressure value is transmitted between the blood pressure measuring device and blood pressure calculating device. This allows for the use of a low bandwidth low costs transmitter and other communication link components without compromising data transfer speed.

The blood-pressure calculating device is a standalone portable device. It uses a plethysmograph that measures a change in arterial blood volume induced by a heart beat. A processor calculates a second blood pressure of the patient using a mathematical model that includes the change in arterial blood volume induced by heart beat and the measured blood pressure value.

Further aspects of the invention will become apparent from the following description which is given by way of example only.

### BRIEF DESCRIPTION OF THE DRAWINGS

FIG. 1 is a schematic illustration of apparatus according to the invention for determining blood pressure of a person.

### DETAINED DESCRIPTION OF THE INVENTION

A method for measuring blood pressure with automatic compensation is described in co-pending U.S. patent application Ser. No. 11/134,637, which was published under the number US 2005/0261593 on Nov. 24, 2005. The entire contents of US 2005/0261593 are explicitly incorporated herein by reference. The current invention will be described as embodied in an apparatus that automatically calibrates a blood pressure calculating device using a method according to co-pending U.S. patent application Ser. No. 11/134,637 however this is not intended to limit the scope of use or functionality of the invention. The skilled addressee with appreciate that the invention will find general application in apparatus for determining the arterial blood pressure of a human.

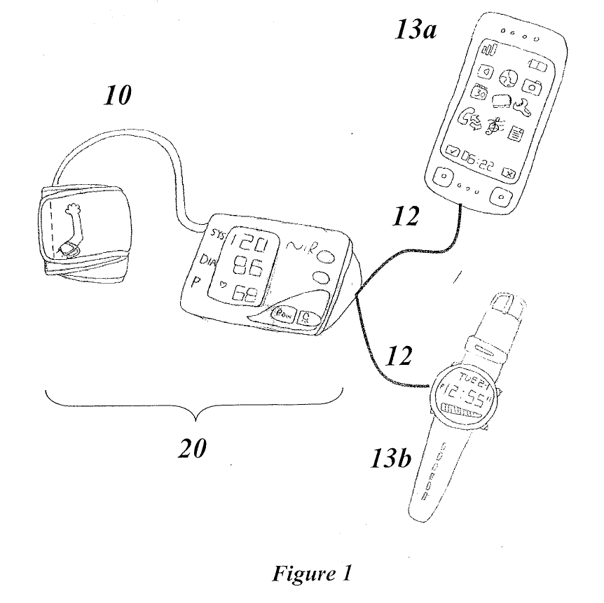

Referring to FIG. 1, an apparatus according to the invention for determining blood pressure of a person includes a conventional cuff based sphygmomanometer blood pressure measuring device 20 comprising an inflatable cuff 10 and a control unit 11 connected to the cuff by an inflation tube and sensor cables. The control unit 11 contains a pump for inflating the cuff 10 when wrapped around a person's arm and various sensors for determining systolic and diastolic blood pressure of the person in automatic fashion using known means.

The control unit 11 also includes a storage medium for storing the measure blood pressure values and a communication device for communicating with a portable blood pressure calculating device 13a, 13b. In the preferred embodiment the communication means is a transmitter connected to an output port which can receive a plug from a cable 12 connected to the portable blood pressure calculating device 13 in order to transfer blood pressure values to the portable device 13. If the control unit 11 is mains power based then the output port and cable 12 can also be used to charge the portable device 13. In an alternative embodiment the communication means may be a transmitter and wireless radio for wireless communication between the control unit 11 and portable blood pressure calculating device 13. The wireless transmission range need only be relatively small, for example only within a few meters, so that a relatively inexpensive radio may be used. Further, and as will be described later, the only data transfer from the control unit 11 to the portable blood pressure calculating means 13 is the measured blood pressure value, for example systolic and/or diastolic blood pressure values, obtained and stored by the blood pressure measuring device 20.

In the preferred embodiment the portable blood pressure calculating device 13 is one that calculates blood pressure using a method such a pulse transmit time (PTT). A full discussion on a method of calculating blood pressure using pulse transmit time can be found in published papers: Messrs J. C. Bramwell and A. V. Hill, “The Velocity of the Pulse Wave in Man”, Proceedings of the Royal Society, London, pp. 298-306, 1922; and in B. Gribbin, A. Steptoe, and P. Sleight, “Pulse Wave Velocity as a Measure of Blood Pressure Change”, Psychophysiology, Vol. 13, No. 1, pp. 86-90, 1976; and in co-pending application published as US 2005/0261593. The portable blood pressure calculating device 13 itself may be of any convenient form such as a small handheld device 13a of the size and shape a palm computer or PDA, or may be a wearable device such as a wristwatch 13b. The portable blood pressure calculating device 13 includes sensors as known in the art for determining perimeters, such as electrocardiogram (ECG) and photoplethysmogram (PPG) signals, needed to calculate pulse transit time of a person.

Because the estimation of blood pressure from pulse transfer time varies with the physiological state of the patient, the portable device 13 must be recalibrated on a regular basis. In the current invention recalibration occurs automatically whenever the portable device 13 is brought within communication range of the control unit 11 from the blood pressure measuring device. The control unit 11 automatically detects the presence of the portable device 13 and transmits the most recently stored blood pressure values of the person to the portable device 13. The patient may, for example check, their blood pressure every morning with the blood pressure measuring device in which new blood pressure values are stored in the control unit 11. The new blood pressure values are then transmitted to the portable device 13 which the patient can carry with them throughout the day for periodic or continuous monitoring of blood pressure.

Those skilled in the art should understand that the above embodiments are only utilized to describe the invention. There are many modifications and variations to the invention without departing the spirit of the invention.

1. An apparatus for determining blood pressure of a person, comprising:
a blood pressure measuring device that measures a blood pressure value of a patient at a first time,
a storage device that stores the blood pressure value that is measured,
a blood pressure calculating device; and
a transmitter device that automatically detects presence of the blood pressure calculating device and transmits the blood pressure value that is measured and stored to the blood-pressure calculating device at a second time.

2. The apparatus of claim 1 wherein the blood pressure measuring device includes an inflatable cuff that restricts blood flow for measuring the blood pressure value.

3. The apparatus of claim 1 wherein the blood-pressure calculating device measures change in arterial blood induced by a heart beat, the blood pressure calculating device including a receiver that receives the blood pressure value that is measured and transmitted and a second storage device that stores the blood pressure value that is measured in the blood pressure calculating device.

4. The apparatus of claim 3 wherein the blood pressure calculating device includes a plethysmograph that measures change in arterial blood volume induced by a heart beat.

5. The apparatus of claim 3 wherein the blood pressure calculating device further includes a processor that calculates blood pressure of the patient at a third time using a mathematical model that includes the change in arterial blood induced by a heart beat and the blood pressure value that is measured.

6. The apparatus of claim 3 wherein the blood pressure calculating device further includes a processor that determines pulse transit time of an arterial pressure wave induced by a heart beat at a third time, and that calculates blood pressure of the patient using a mathematical model that includes the pulse transmit time determined at the third time and the blood pressure value that is measured and calculated at the first time.

7. The apparatus of claim 3 wherein the blood pressure calculating device is a battery operated portable device.

8. The apparatus of claim 1 wherein the blood pressure measuring device does not include a processor that uses a mathematical model or calculation that includes a change in arterial blood induced by heart beat and the blood pressure value that is measured.

9. The apparatus of claim 1 wherein only the blood pressure value that is measured is transmitted between the blood pressure measuring device and the blood pressure calculating device.

10. The apparatus of claim 1 wherein the blood pressure value that is measured consists of systolic and/or diastolic blood pressure.
